(19) United States
(12) Patent Application Publication
(10) Pub. No.: US 2016/0097100 A1
(43) Pub. Date: Apr. 7, 2016

(54) GENETIC TEST TO PREDICT PATIENT RESPONSE TO BONE MORPHOGENETIC PROTEIN IN ARTHRODESIS

(71) Applicants: Jeffrey TRENT, Phoenix, AZ (US); Clifford Barry JONES, East Grand Rapids, MI (US); Debra Lynn SIETSEMA, Byron Center, MI (US); THE TRANSLATIONAL GENOMICS RESEARCH INSTITUTE, Phoenix, AZ (US); ORTHOPAEDIC ASSOCIATES OF MICHIGAN, Grand Rapids, MI (US)

(72) Inventors: Jeffrey TRENT, Phoenix, AZ (US); Clifford Barry JONES, East Grand Rapids, MI (US); Debra Lynn SIETSEMA, Byron Center, MI (US)

(21) Appl. No.: 14/891,693

(22) PCT Filed: May 19, 2014
(86) PCT No.: PCT/US14/38676

§ 371 Date: Nov. 17, 2015

Related U.S. Application Data
(60) Provisional application No. 61/824,916, filed on May 17, 2013.

(51) Int. Cl. C12Q 1/68

(52) U.S. Cl. CPC C12Q 1/6883; C12Q 2600/156; C12Q 2600/106; C12Q 2600/172

(57) ABSTRACT

The present invention provides a method of predicting an increased likelihood of a complication associated with administration of a bone morphogenetic protein (BMP) to a subject in need of a spinal fusion surgery by detecting single nucleotide polymorphisms (SNPs) in the subject. Also provided are methods of treatment with a BMP based on the presence or absence of SNPs and kits for use in detecting the SNPs.

### CROSS REFERENCE TO RELATED APPLICATIONS

This application claims priority to U.S. Provisional Application Ser. No. 61/824,916, filed on May 17, 2013. The contents of this reference are hereby incorporated by reference in their entirety.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 3,388 kilobyte ASCII (text) file named “BMP_ST25” created on May 16, 2014.

### FIELD OF THE INVENTION

This application relates to the field of medical diagnostics and the use of single nucleotide polymorphisms in predicting an increased likelihood of a complication associated with administration of a bone morphogenetic protein to a subject undergoing a spinal fusion surgery. The application is also directed to methods of treatment guided by the detection of one or more single nucleotide polymorphisms in a subject.

### BACKGROUND OF THE INVENTION

Currently there are nearly 500,000 lumbar and cervical spine fusion procedures performed each year in the United States. One of the causes of back pain and disability results from the rupture or degeneration of one or more intervertebral discs in the spine. Surgical procedures are commonly performed to correct problems with displaced, damaged, or degenerated intervertebral discs due to trauma, disease, or aging. Furthermore, aging of the spine can cause bony spurs and joints swelling (hypertrophy) which can compress and impinge on the spinal cord and/or nerve roots (stenosis). Generally, spinal fusion procedures involve removing some or the all of the diseased or damaged disc, and inserting one or more intervertebral implants into the resulting disc space (interbody fusion). It also involves removal of the stenotic tissue impinging the neural structures and placing bone graft material posteriorly adjacent to the transverse processes (posterolateral fusion).

Minimally invasive methods of performing spinal fusion have gained popularity in recent years due to the many benefits of the procedure which include diminished dissection of body tissue and lower blood loss during surgery resulting in reduced surgery time, lower post-operative pain, and potentially a quicker recovery for patients.

Current spinal fusion implants utilize grafts of either bone or artificial implants to fill the intervertebral disc space or posteriorly adjacent to the transverse process. Spinal fusion implants or grafts may be made of metal, plastic composites, ceramics, or bone. Natural bone grafts have also been developed including autologous and allograft bone grafts. Other bone grafts may include certain man-made substances including binder joining bone chips and composite bone structures. Further, graft material placed posteriorly can consist of autograft, allograft, and demineralized bone materials (DBM).

While generally effective, the use of bone grafts presents several disadvantages. Autologous bone grafts are obtained from bone material surgically removed from the iliac crest of a patient. This method can be detrimental because it may not yield a sufficient quantity of graft material, requires additional surgery and time, and increases the risk of infection, blood loss, and neural injury. Moreover, the structural integrity at the donor site can be reduced and significant morbidity such as a fracture associated with harvesting the autologous bone graft may occur. Bone graft donor sites result in varying levels of chronic pain and disability.

Allograft bone grafts are obtained from cadaveric specimens, machined, and sterilized for implantation. Production of allograft bone implants may be difficult because of the inherent challenges in forecasting the receipt of cadavers. Allograft may also only provide temporary support, as it is difficult to manufacture the allograft with consistent shape and strength given the differing characteristics of cadavers. Besides structure, allograft provides no stimulus to create bone or enhance fusions.

Bone-morphogenetic proteins (BMPs) represent a family of differentiation factors that promote bone creation and remodeling. Urist et al., (1984) Proc Natl Acad Sci USA 81:371-375. Clinical use of recombinant BMP protein was approved by the US Food and Drug Administration (FDA) in 2002 for surgery of the anterior lumbar spine to promote bone fusion. Two BMP products are commercially available for clinical use, BMP-2 (INFUSE, Medtronic, Memphis, Tenn.) and BMP-7 (OP-1 Putty, Stryker, Kalamazoo, Mich.). BMP-2 is approved for anterior lumbar interbody fusion in skeletally mature patients and BMP-7 received a humanitarian use device approval in 2003 for revision intertransverse lumbar fusion in compromised patients. Due to robust bone forming properties, BMP use may increase the likelihood of bony fusion thereby decreasing the undesired outcome of pseudarthrosis or nonunion. Burkus et al., (2005) J Bone Joint Surg Am. 87:1205-1212. An additional clinical benefit relates to the decreased morbidity from bone-graft harvest because solid fusion may be achieved without the need for autologous graft. Dimar et al., (2006) Spine 31:2534-2539.

Although BMP is FDA approved for use only in the lumbar spine, recent work has focused on applications at other spine levels, including use in cervical interbody fusion. Many surgeons utilize BMP's in an “off-label” method for posterolateral fusions in conjunction with allograft or other bone void filler to provide bulk (See Bone Morphogenetic Protein: The State of the Evidence for On-Label and Off-Label Use: Disposition of Comments. September 2012. Agency for Healthcare Research and Quality, Rockville, Md. and On- and Off-label Uses of rhBMP-2 or rhBMP-7 for Spinal Fusion, Health Technology Assessment, Feb. 14, 2012. Washington State Health Care Authority, Olympia, Wash.). BMP has been utilized for posterolateral fusion in an off label fashion with excellent results related to fusion but with varying levels of complications. Hoffmann et al., (2012) Arch Orthop Trauma Surg 132:1105-1110; Hoffmann et al., (2013) J Orthop Surg Res 8:1-6. Complications following use of BMP in humans have been reported for both lumbar as well as cervical fusion procedures. Complications can include hyperemia induced swelling, seroma formation compressing neural structures, and excess bone formation. Currently the FDA has banned the use of BMP for anterior cervical interbody fusions because of the excess swelling associated with the physiological response to BMP resulting in life threatening postoperative swelling and breathing occlusion. See FDA Public Health Notification: Life-threatening Complications Associated with Recombinant Human Bone Morphogenetic Protein in Cervical Spine Fusion, Jul. 1, 2008.

There is a need for pre-operative genetic screening to guide BMP use in spinal arthrodesis patients to identify those patients at risk of developing complications. The present invention is directed to addressing this need by detecting single nucleotide polymorphisms that are predictive of a risk of complication associated with BMP administration.

### SUMMARY

Some embodiments of the present invention provide a method of predicting an increased likelihood of a complication associated with administration of a BMP in a human subject in need of a spinal fusion surgery. The method may include obtaining a sample from the human subject in need of the spinal fusion surgery, detecting in the sample the presence of one or a combination of two or more single nucleotide polymorphisms (SNPs) selected from the group comprising a thymine at the polymorphic position of rs10733133 (SEQ ID NO: 1), a cytosine at the polymorphic position of rs1126933 (SEQ ID NO: 2), a thymine at the polymorphic position of rs2433031 (SEQ ID NO: 3), a guanine at the polymorphic position of rs6571751 (SEQ ID NO: 4), and a thymine at the polymorphic position of rs7318267 (SEQ ID NO: 5). The method may also include predicting that the human subject with the one or combination of SNPs has an increased likelihood of experiencing a complication resulting from administration of a BMP compared to a human subject without the one or combination of SNPs.

In some aspects, the method of the present invention may further comprise the steps of amplifying a nucleic acid sequence comprising the polymorphic position of an SNP selected from the group consisting of rs10733133 (SEQ ID NO: 1), rs1126933 (SEQ ID NO: 2), rs2433031 (SEQ ID NO: 3), rs6571751 (SEQ ID NO: 4), and rs7318267 (SEQ ID NO: 5) using a first primer that binds upstream of said position and a second primer that binds downstream of said position and determining the genotype of the human subject at said position. In certain aspects, the BMP may be BMP2, BMP3, BMP4, BMP5, BMP6, BMP7, BMP8a, BMP8b, BMP9, BMP10 and BMP15. In other aspects, the BMP may be recombinant human bone morphogenetic protein-2 (rhBMP-2) or recombinant human bone morphogenetic protein-7 (rhBMP-7).

The present invention also includes a method of treating a human subject in need of a spinal fusion surgery comprising obtaining a sample from the human subject in need of the spinal fusion surgery. The method may include detecting in the sample the absence of one or a combination of two or more SNPs selected from the group consisting of a thymine at the polymorphic position of rs10733133, a cytosine at the polymorphic position of rs1126933, a thymine at the polymorphic position of rs2433031, a guanine at the polymorphic position of rs6571751, and a thymine at the polymorphic position of rs7318267, wherein the absence of the one or combination of SNPs indicates a decreased likelihood of experiencing a complication associated with administration of a BMP compared to a human subject with the one or combination of SNPs. Thereafter, the method may include administering a BMP to the human subject.

In other embodiments, the present invention includes a method of treating a human subject in need of a spinal fusion surgery comprising obtaining a sample from the human subject in need of a spinal fusion surgery and detecting in the sample the presence of one or a combination of two or more SNPs selected from the group consisting of a cytosine at the polymorphic position of rs10733133, a guanine at the polymorphic position of rs1126933, an adenine at the polymorphic position of rs2433031, an adenine at the polymorphic position of rs6571751, and a cytosine at the polymorphic position of rs7318267. In particular, the presence of the one or combination of SNPs indicates a decreased likelihood of experiencing a complication associated with administration of a BMP compared to a human subject with the one or combination of SNPs. In some aspects, the method may include administering a BMP to the human subject. For example, the BMP may be administered before, during, or after the spinal fusion surgery.

The present invention includes a kit for predicting an increased likelihood of a complication associated with administration of a BMP in a human subject in need of a spinal fusion surgery comprising at least one probe or pair of primers for the amplification and/or detection of one or a combination of two or more single nucleotide polymorphisms (SNPs) selected from the group consisting of a thymine at the polymorphic position of rs10733133 (SEQ ID NO: 1), a cytosine at the polymorphic position of rs1126933 (SEQ ID NO: 2), a thymine at the polymorphic position of rs2433031 (SEQ ID NO: 3), a guanine at the polymorphic position of rs6571751 (SEQ ID NO: 4), and a thymine at the polymorphic position of rs7318267 (SEQ ID NO: 5). In certain embodiments, the kit includes a polymerizing agent and chain elongating nucleotides.

Also included in the present invention is an apparatus for predicting an increased likelihood of a complication associated with administration of a BMP in a human subject in need of a spinal fusion surgery comprising a substrate and at least two oligonucleotides bound to the substrate, wherein each of the at least two oligonucleotides comprises a contiguous nucleic acid sequence complementary to a different sequence selected from group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15. In certain embodiments, the substrate may be a glass slide and the apparatus may comprise a microarray.

Additional objectives, advantages and novel features will be set forth in the description which follows or will become apparent to those skilled in the art upon examination of the drawings and detailed description which follows.

### BRIEF DESCRIPTION OF THE DRAWINGS

FIG. 1 is bar graph showing a joint effect plot detailing the number of risk alleles present from the 5 single nucleotide polymorphisms of interest (x-axis) within the hyper-responders as compared to non-hyper-responders (y-axis).

### DETAILED DESCRIPTION

As used herein, the verb “comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”.

As used herein, a “sample,” such as a biological sample that includes nucleic acid molecules, is a sample obtained from a subject. As used herein, biological samples include all clinical samples including, but not limited to, cells, tissues, and bodily fluids, such as: blood; derivatives and fractions of blood, such as serum; extracted galls; biopsied or surgically removed tissue, including tissues that are, for example, unfixed, frozen, fixed in formalin and/or embedded in paraffin; tears; milk; skin scrapes; surface washings; urine; sputum; cerebrospinal fluid; prostate fluid; pus; or bone marrow aspirates. In a particular example, a sample includes blood obtained from a human subject, such as whole blood or serum. In another particular example, a sample includes buccal cells, for example collected using a swab or by an oral rinse.

As used herein, the term “polynucleotide”, “nucleic acid” or “nucleic acid molecule” refers to a polymer composed of nucleotide units, including naturally occurring nucleic acids, such as deoxyribonucleic acid (“DNA”) and ribonucleic acid (“RNA”) as well as nucleic acid analogs including those which have non-naturally occurring nucleotides. Polynucleotide can be synthesized, for example, using an automated DNA synthesizer. The term “nucleic acid” or “nucleic acid molecule” typically refers to a large polynucleotide. It will be understood that when a nucleic acid fragment is represented by a DNA sequence (i.e., A, T, G, C), this also includes an RNA sequence (i.e., A, U, G, C) in which “U” replaces “T.”

As used herein, the term “allele” refers to variants of a nucleotide sequence. A biallelic polymorphism has two forms. Diploid organisms may be homozygous or heterozygous for an allelic form.

As used herein, the term “risk allele” refers to variants of a nucleotide sequence that are predictive of an increased likelihood of a human subject experiencing a complication resulting from administration of a BMP compared to a human subject without the risk allele. Risk alleles include a thymine at the polymorphic position of rs10733133 (SEQ ID NO: 1), a cytosine at the polymorphic position of rs1126933 (SEQ ID NO: 2), a thymine at the polymorphic position of rs2433031 (SEQ ID NO: 3), a guanine at the polymorphic position of rs6571751 (SEQ ID NO: 4), and a thymine at the polymorphic position of rs7318267 (SEQ ID NO: 5).

As used herein, the term “single nucleotide polymorphism” (aka “SNP”) refers to single nucleotide polymorphisms in DNA. SNPs are usually preceded and followed by highly conserved sequences that vary in less than 1/100 or 1/1000 members of the population. An individual may be homozygous or heterozygous for an allele at each SNP position. An SNP may, in some instances, be referred to as a “cSNP” to denote that the nucleotide sequence containing the SNP is an amino acid “coding” sequence. A SNP may arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. For example, if at a particular chromosomal location, one member of a population has an adenine (A) and another member of the population has a cytosine (C) at the same position, then this position is a SNP. Alleles for SNP markers as referred to herein are expressed by the bases A, C, G or T as they occur at the polymorphic site in the SNP assay employed.

The nomenclature of SNPs as described herein refers to the official Reference SNP (rs) ID identification tag as assigned to each unique SNP by the National Center for Biotechnological Information (NCBI). The NCBI database of SNPs is accessible to the public online.

As used herein, the term “genotype” means the identification of the alleles present in an individual or a sample. The term “genotyping” a sample or an individual for a genetic marker may comprise determination of which allele or alleles an individual carries for one or more SNPs. For example, a particular nucleotide in a genome may be a T in some individuals and a C in other individuals. Those individuals who have a T at the position have the T allele and those who have a C have the C allele. In a diploid organism the individual will have two copies of the sequence containing the polymorphic position. So the individual may have a T allele and a C allele, or alternatively two copies of the T allele, or two copies of the C allele. Each allele may be present at a different frequency in a given population. Those individuals who have two copies of the C allele are homozygous for the C allele and the genotype is CC, those individuals who have two copies of the T allele are homozygous for the T allele and the genotype is TT, and those individuals who have one copy of each allele are heterozygous and the genotype is TC.

As used herein, the term “primer” refers to a specific oligonucleotide sequence which is complementary to a target nucleotide sequence and used to hybridize to the target nucleotide sequence. A primer serves as an initiation point for nucleotide polymerization catalyzed by DNA polymerase, RNA polymerase or reverse transcriptase.

As used herein, the term “probe” refers to a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified.

As used herein, a “bone morphogenetic protein” or a “BMP” is an extracellular signaling molecule that belongs to the transforming growth factor-β (TGF-β) superfamily and may be any one of BMP2, BMP3, BMP4, BMP5, BMP6, BMP7, BMP8a, BMP8b, BMP9, BMP10 and BMP15. Although BMPs were originally identified as factors involved in the formation of bone and cartilage tissue, they have been shown to demonstrate a wide range of biological effects. BMPs affect structures and processes throughout the entire body, including embryonic patterning and development, tissue homeostasis and regeneration, and stem cell maintenance, function and environment (Varga et al., (2005); Oncogene 24:5713-5721; Wagner, (2007), FEBS J. 274:2968-2976; Miyazono et al., (2010), J. Biochem. 147:35-51). Furthermore, BMPs have been shown to regulate proliferation, differentiation, and apoptosis in many different cell types by modulating the transcription of specific target genes.

At the molecular level, BMPs transduce their signals primarily through a heterotetrameric complex comprising transmembrane type I and type II serine/threonine kinase receptors. In mammals there are three type I receptors and three type II receptors that have been identified for BMPs. They include type I receptors BMPR1A (ALK-3), BMPR1B (ALK-6) and ACVR1A (ALK-2) and type II receptors BMPR2 (BMPR-II), ACVR2A (ACTRII or ACTRIIA) and ACVR2B (ACTRIIB).

Evidence has shown that both type I and type II receptors are required for signal transduction. Upon ligand binding, constitutively active type II receptors phosphorylate type I receptors, triggering activation of the type I receptor and subsequent intracellular SMAD signal transduction cascades. For example, an activated type I receptor phosphorylates intracellular receptor-associated SMADs (SMAD-1, SMAD-5 and/or SMAD-8) which allows SMAD-1, 5, 8 to interact with common partner SMAD4. This complex of SMADs translocates to the nucleus and regulates gene transcription of target genes, including proteins such as p21/Cip1/Waf1, bax, p53, Id1-3, OASIS, Prx2, TIEG, Snail, Hey 1 and Tcf7. (See e.g., Massague, (1998) Annu. Rev. Biochem. 67:753-791; Miyazono et al., (2010), J. Biochem. 147:35-51.)

As used herein, a “spinal fusion surgery” (also referred to herein as “arthrodesis”) is a surgical technique that joins two or more vertebrae. A spinal fusion surgery may be a posterolateral fusion or an interbody fusion. Specific types of interbody fusion include anterior lumbar interbody fusion (ALIF), posterior lumbar interbody fusion (PLIF), transforaminal lumbar interbody fusion (TLIF), transpsoas interbody fusion (DLIF or XLIF), and cervical interbody fusion.

As used herein, a “complication” is an adverse effect on the health of a human subject. A complication may result from a spinal fusion surgery and/or from administration of a BMP. A complication may be, without limitation, any one of the following: a postoperative formation of a sterile seroma, painful edema, a compressive fluid collection, a bone overgrowth, a reoperation secondary to failed symptomatic fusion, an infection, bleeding, pseudoarthrosis, nerve damage, a blood clot, an injury to a blood vessel in or around the spine, and hyperemia induced swelling.

The term “hyperresponse” as used herein refers to a hyper reaction to a therapeutic agent such that the subject to whom the therapeutic agent is administered experiences one or more resulting complications. A subject with a hyperresponse to a BMP experiences complications such as, but not limited to, seroma formation, painful edema, compressive fluid collection, bone overgrowth, and/or hyperemia induced swelling.

An “increased likelihood,” as used herein, refers to an increased probability that a condition will be present in a subject compared to the occurrence of the condition in a control or reference subject. The condition may be a complication associated with administration of a BMP. The increased probability may be an increase of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% that the condition will be present in the subject compared to the occurrence of the condition in a control or reference subject. The increased probability may also be an increase of 1% to 100%; e.g., any range within 1% to 100%, such as 5% to 85%, 10% to 70%; 20% to 60%, 30% to 50%, etc. over that observed with a control or reference subject.

A “decreased likelihood,” as used herein, refers to a decreased probability that a condition will be present in a subject compared to the occurrence of the condition in a control or reference subject. The condition may be a complication associated with administration of a BMP. The decreased probability may be a decrease of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% that the condition will be present in the subject compared to the occurrence of the condition in a control or reference subject. The decreased probability may also be a decrease of 1% to 100%; e.g., any range within 1% to 100%, such as 5% to 85%, 10% to 70%; 20% to 60%, 30% to 50%, etc. compared to that observed with a control or reference subject.

The expression of the biomarker, allele and/or SNPs (these terms are used interchangeably herein) in a sample may be compared to a level of expression predetermined to predict the presence or absence of a particular physiological characteristic. The level of expression may be derived from a single control or a set of controls. A control may be any sample with a previously determined level of expression. A control may comprise material within the sample or material from sources other than the sample. Alternatively, the expression of a biomarker in a sample may be compared to a control that has a level of expression predetermined to signal or not signal a cellular or physiological characteristic. This level of expression may be derived from a single source of material including the sample itself or from a set of sources. Comparison of the expression of the biomarker in the sample to a particular level of expression results in a prediction that the sample exhibits or does not exhibit the cellular or physiological characteristic.

In addition, one or more levels of expression of the biomarker may be selected that provide an acceptable ability of its ability to signify a particular physiological or cellular characteristic. Examples of such characteristics include identifying or diagnosing a particular disease, assessing a risk of outcome or a prognostic risk, or assessing the risk that a particular treatment will or will not be effective.

For example, Receiver Operating Characteristic curves, or “ROC” curves, may be calculated by plotting the value of a variable versus its relative frequency in two populations. For any particular biomarker, a distribution of biomarker expression levels for subjects with and without a disease may overlap. This indicates that the test does not absolutely distinguish between the two populations with complete accuracy. The area of overlap indicates where the test cannot distinguish the two groups. A threshold is selected. Expression of the biomarker in the sample above the threshold indicates the sample is similar to one group and expression of the biomarker below the threshold indicates the sample is similar to the other group. The area under the ROC curve is a measure of the probability that the expression correctly indicated the similarity of the sample to the proper group. See, e.g., Hanley et al., Radiology 143: 29-36 (1982) hereby incorporated by reference.

Additionally, levels of expression may be established by assessing the expression of a biomarker in a sample from one patient, assessing the expression of additional samples from the same patient obtained later in time, and comparing the expression of the biomarker from the later samples with the initial sample or samples. This method may be used in the case of biomarkers that indicate, for example, progression or worsening of disease or lack of efficacy of a treatment regimen or remission of a disease or efficacy of a treatment regimen.

Other methods may be used to assess how accurately the expression of a biomarker signifies a particular physiological or cellular characteristic. Such methods include a positive likelihood ratio, negative likelihood ratio, odds ratio, and/or hazard ratio. In the case of a likelihood ratio, the likelihood that the expression of the biomarker would be found in a sample with a particular cellular or physiological characteristic is compared with the likelihood that the expression of the biomarker would be found in a sample lacking the particular cellular or physiological characteristic.

An odds ratio measures effect size and describes the amount of association or non-independence between two groups. An odds ratio is the ratio of the odds of a biomarker being expressed in one set of samples versus the odds of the biomarker being expressed in the other set of samples. An odds ratio of 1 indicates that the event or condition is equally likely to occur in both groups. An odds ratio grater or less than 1 indicates that expression of the biomarker is more likely to occur in one group or the other depending on how the odds ratio calculation was set up.

A hazard ratio may be calculated by estimate of relative risk. Relative risk is the chance that a particular event will take place. It is a ratio of the probability that an event such as development or progression of a disease will occur in samples that exceed a threshold level of expression of a biomarker over the probability that the event will occur in samples that do not exceed a threshold level of expression of a biomarker. Alternatively, a hazard ratio may be calculated by the limit of the number of events per unit time divided by the number at risk as the time interval decreases. In the case of a hazard ratio, a value of 1 indicates that the relative risk is equal in both the first and second groups. A value greater or less than 1 indicates that the risk is greater in one group or another, depending on the inputs into the calculation.

Additionally, multiple threshold levels of expression may be determined. This can be the case in so-called “tertile,” “quartile,” or “quintile” analyses. In these methods, multiple groups can be considered together as a single population, and are divided into 3 or more bins having equal numbers of individuals. The boundary between two of these “bins” may be considered threshold levels of expression indicating a particular level of risk of a disease developing or signifying a physiological or cellular state. A risk may be assigned based on which “bin” into which a subject can be categorized.

Methods for predicting an increased likelihood of a complication associated with administration of a BMP in a subject include detecting the presence or absence of one or more of the polymorphisms described herein in a human nucleic acid sample.

Nucleic acids for diagnosis may be obtained from a patient's cells, such as from blood, serum, plasma, urine, saliva, or a tissue biopsy. The nucleic acid sample can be isolated from a biological sample using standard techniques. The nucleic acid sample may be isolated from the subject and then directly utilized in a method for determining the presence of a polymorphic variant, or alternatively, the sample may be isolated and then stored (e.g., frozen) for a period of time before being subjected to analysis.

In some embodiments, the present invention is directed to a method of predicting an increased likelihood of a complication associated with administration of a BMP in a human subject in need of a spinal fusion surgery comprising the steps of: (i) obtaining a sample from the human subject in need of a spinal fusion surgery; (ii) detecting in the sample the presence of one or a combination of two or more single nucleotide polymorphisms (SNPs) selected from the group consisting of a thymine at the polymorphic position of rs10733133 (SEQ ID NO: 1), a cytosine at the polymorphic position of rs1126933 (SEQ ID NO: 2), a thymine at the polymorphic position of rs2433031 (SEQ ID NO: 3), a guanine at the polymorphic position of rs6571751 (SEQ ID NO: 4), and a thymine at the polymorphic position of rs7318267 (SEQ ID NO: 5); and (iii) predicting the human subject with the one or combination of SNPs has an increased likelihood of experiencing a complication associated with administration of a BMP compared to a human subject without the one or combination of SNPs.

In certain aspects, the complication results from a hyperresponse to a BMP. The hyperresponse may occur during a spinal fusion surgery or at any time subsequent to the spinal fusion surgery. The hyperresponse may cause complications such as seroma formation, painful edema, compressive fluid collection, bone overgrowth, and hyperemia induced swelling. These complications can result in damage to the surrounding tissue, discomfort, pain, slowed healing, and infection.

In other aspects, the human subject may be heterozygous for at least one of the SNPs of the present invention. In other aspects, the human subject may be homozygous for at least one of the SNPs of the present invention.

In some embodiments, one, two, three, four, five, six, seven, eight, nine, ten, or more risk alleles may be detected in the sample from the human subject. As the number of risk alleles increases in the sample from the human subject, the risk of developing a complication associated with administration of a BMP typically increases as well.

Risk alleles are shown at the polymorphic positions in Table 1, which also presents the naturally occurring nucleotide sequences from Homo sapiens containing rs10733133 (SEQ ID NO: 1), rs1126933 (SEQ ID NO: 2), rs2433031 (SEQ ID NO: 3), rs6571751 (SEQ ID NO: 4), and rs7318267 (SEQ ID NO: 5). These sequences were obtained from the NCBI's database wherein the nucleotide within the brackets is the polymorphic nucleotide. The polymorphic nucleotides of rs10733133, rs1126933, rs2433031, rs6571751, and rs7318267 are located at position 27 of SEQ ID NO: 1, SEQ ID NO: 2, SEQ ID NO: 3, SEQ ID NO: 4, and SEQ ID NO: 5, respectively.

TABLE 1
Single nucleotide polymorphisms (SNPs) that are
predictive of the occurrence of a complication
associated with administration of a BMP.

| SNP | Nucleotide Sequence |
|---|---|
| rs10733133 | GAAAGCATACTTGGCCAAATTTTCAA[C/T]TGATTC |
| | CCTGTCATTAGCAAATATG (SEQ ID NO: 1) |
| rs1126933 | ATCTGCAACCAAAGCCCTGTGCGCCA[C/G]GTGCTG |
| | AACATCACGGACAAGGACC (SEQ ID NO: 2) |
| rs2433031 | TTAGAACACTCTGTATTAAGCCAGCA[A/T]TGTGAG |
| | TATAAAATGATTTCTAAAT (SEQ ID NO: 3) |
| rs6571751 | ATGAAAACAGAGGTGAAGTAGCCAGT[A/G]AACCCA |
| | GTGAACTGTGAGTTCTTCT (SEQ ID NO: 4) |
| rs7318267 | GTCCTCATCCTCCTTCCTCAAAGCCA[C/T]CGGCTC |
| | CTCCTGGACGGGTTGGGTA (SEQ ID NO: 5) |

The relevant gene information corresponding to each of the SNPs is presented in Table 2. SNP rs10733133 is located within the NID1 gene. NID1 (aka nidogen 1 and NID) encodes a member of the nidogen family of basement membrane glycoproteins. The protein interacts with several other components of basement membranes, and may play a role in cell interactions with the extracellular matrix. SNP rs1126933 is found within the CDH3 gene. CDH3 (aka cadherin 3, type 1, P-cadherin (placental); CDHP; HJMD; and PCAD) is a classical cadherin from the cadherin superfamily. The encoded protein is a calcium-dependent cell-cell adhesion glycoprotein comprised of five extracellular cadherin repeats, a transmembrane region and a highly conserved cytoplasmic tail. This gene is located in a six-cadherin cluster in a region on the long arm of chromosome 16 that is involved in loss of heterozygosity events in breast and prostate cancer. In addition, aberrant expression of this protein is observed in cervical adenocarcinomas. Mutations in this gene have been associated with congenital hypotrichosis with juvenile macular dystrophy.

SNP rs2433031 is located within the SENP7 gene. SENP7 is also known as SUMO1/sentrin specific peptidase 7. The reversible posttranslational modification of proteins by the addition of small ubiquitin-like SUMO proteins is required for many cellular processes. SUMO-specific proteases, such as SENP7, process SUMO precursors to generate a C-terminal diglycine motif required for the conjugation reaction. They also display isopeptidase activity for deconjugation of SUMO-conjugated substrates. SNP rs6571751 is found within the RPGRIP1 gene. RPGRIP1 (aka retinitis pigmentosa GTPase regulator interacting protein 1, LCA6, RGI1, RGRIP, CORD13, RPGRIP, and RPGRIP1d) encodes a photoreceptor protein that interacts with retinitis pigmentosa GTPase regulator protein and is a key component of cone and rod photoreceptor cells. Mutations in this gene lead to autosomal recessive congenital blindness. SNP rs7318267 is present in the FARP1 gene. FARP1 (aka FERM, RhoGEF (ARHGEF) and pleckstrin domain protein 1 (chondrocyte-derived); CDEP; PLEKHC2; and PPP1R75) was originally isolated through subtractive hybridization due to its increased expression in differentiated chondrocytes versus dedifferentiated chondrocytes. The resulting protein contains a predicted ezrin-like domain, a Dbl homology domain, and a pleckstrin homology domain. It is believed to be a member of the band 4.1 superfamily whose members link the cytoskeleton to the cell membrane. Two alternatively spliced transcript variants encoding distinct isoforms have been found for this gene.

TABLE 2
Relevant gene information corresponding to the five SNPs and
the type of genetic variation introduced by each.

| | | | Gene | Type of Genetic |
|---|---|---|---|---|
| SNP | Chromosome | BP | Name | Variation |
| rs10733133 | 1q43 | 23620877 | NID1 | nonsynonymous |
| | | | | substitution |
| rs1126933 | 16q22.1 | 68721533 | CDH3 | nonsynonymous |
| | | | | substitution |
| rs2433031 | 3q12 | 10106671 | SENP7 | splice modification |
| rs6571751 | 14q11 | 21770730 | RPGRIP1 | nonsynonymous |
| | | | | substitution |
| rs7318267 | 13q32.2 | 98896776 | FARP1 | nonsynonymous |
| | | | | substitution |

The present invention includes a method of treating a human subject in need of a spinal fusion surgery. Typically, the method involves obtaining a sample from the human subject in need of a spinal fusion surgery.

Generally, the sample from the human subject is tested for the either: a) the presence of one or a combination of two or more SNPs selected from the group consisting of a cytosine at the polymorphic position of rs10733133, a guanine at the polymorphic position of rs1126933, an adenine at the polymorphic position of rs2433031, an adenine at the polymorphic position of rs6571751, and a cytosine at the polymorphic position of rs7318267; or b) the absence of one or a combination of two or more SNPs selected from the group consisting of a thymine at the polymorphic position of rs10733133, a cytosine at the polymorphic position of rs1126933, a thymine at the polymorphic position of rs2433031, a guanine at the polymorphic position of rs6571751, and a thymine at the polymorphic position of rs7318267.

The presence or absence of the specified SNPs indicates a decreased likelihood of experiencing a complication associated with administration of a BMP.

Upon detecting the presence or absence of the specified SNPs, BMP may be administered to the human subject. Administration of the BMP may occur prior to, during, and/or after the spinal fusion surgery. Alternatively, if the presence or absence of the specified SNPs is not detected, spinal fusion may occur without administration of a BMP. When a BMP is not administered, typically a graft of either bone or of an artificial implant is inserted into the intervertebral disc space.

In certain embodiments, the BMP is recombinant human bone morphogenetic protein-2 (rhBMP-2) or recombinant human bone morphogenetic protein-7 (rhBMP-7). rhBMP-2 may be administered as part of the INFUSE® Bone Graft product. INFUSE® Bone Graft represents a rhBMP-2 formulation combined with a bovine-derived absorbable collagen sponge (ACS) carrier. To use INFUSE® Bone Graft, surgeons reconstitute the rhBMP-2 powder with sterile water and then apply it to collagen sponges. The sponges are inserted inside a pair of metallic cages, which are then implanted between the vertebrae. The thimble-like cages stabilize the spine and maintain the proper height between the vertebrae while fusion occurs. For posterolateral intertransverse fusions, INFUSE® is applied to the collagen sponge and then added to or wrapped around allograft or other bone void filler to add bulk. This is then placed into the posterolateral gutter adjacent to the transverse processes.

In some embodiments, one or more growth factors of the transforming growth factor-beta 1 (TGF-β1) superfamily are administered to the human subject upon detecting the presence or absence of the specified SNPs. In some embodiments, the one or more growth factors are selected from the group consisting of TGF-β1, bone morphogenetic protein (BMP)-2, BMP-3, BMP-4, BMP-5, BMP-6 and BMP-7. In some embodiments, the one or more growth factors are selected from the group consisting of TGF-β1, BMP-2, BMP-4 and BMP-7. In some embodiments, the one or more growth factors are selected from the group consisting of TGF-β1, BMP-2, BMP-3, BMP-4, BMP-5, BMP-6, BMP-7, fibroblast growth factor-2 (FGF-2), hepatic growth factor (HGF), vascular endothelial growth factor (VEGF) and insulin-like growth factor 1 (IGF-1). In other embodiments, cartilage oligomeric matrix protein (COMP) bound to one or more of the growth factors is administered to the subject.

The method of treatment may further comprise performing a spinal fusion surgery such as a posterolateral fusion, an anterior lumbar interbody fusion (ALIF), a posterior lumbar interbody fusion (PLIF), a transforaminal lumbar interbody fusion (TLIF), a transpsoas interbody fusion (DLIF or XLIF), or a cervical interbody fusion.

Generally, the methods disclosed herein involve an assessment of nucleic acid sequence. Molecular techniques of use in all of these methods are discussed below.

### Preparation of Nucleic Acids for Analysis

Nucleic acid molecules can be prepared for analysis using any technique known to those skilled in the art. Generally, such techniques result in the production of a nucleic acid molecule sufficiently pure to determine the presence or absence of one or more variations at one or more locations in the nucleic acid molecule. Such techniques are described for example, in Sambrook, et al., Molecular Cloning: A Laboratory Manual (Cold Spring Harbor Laboratory, New York) (1989), and Ausubel, et al., Current Protocols in Molecular Biology (John Wiley and Sons, New York) (1997), incorporated herein by reference.

When the nucleic acid of interest is present in a cell, it can be necessary to first prepare an extract of the cell and then perform further steps, such as differential precipitation, column chromatography, extraction with organic solvents and the like, in order to obtain a sufficiently pure preparation of nucleic acid. Extracts can be prepared using standard techniques in the art, for example, by chemical or mechanical lysis of the cell. Extracts then can be further treated, for example, by filtration and/or centrifugation and/or with chaotropic salts such as guanidinium isothiocyanate or urea or with organic solvents such as phenol and/or chloroform to denature any contaminating and potentially interfering proteins. When chaotropic salts are used, it can be desirable to remove the salts from the nucleic acid-containing sample. This can be accomplished using standard techniques in the art such as precipitation, filtration, size exclusion chromatography and the like. In some examples, nucleic acids can be isolated using commercially available kits (e.g., Qiagen, Valencia, Calif.; Life Technologies/Invitrogen, Carlsbad, Calif.; Epicentre, Madison, Wis.).

In some instances, messenger RNA can be extracted from cells. Techniques and material for this purpose are known to those skilled in the art and can involve the use of oligo dT attached to a solid support such as a bead or plastic surface. In some embodiments, the mRNA can be reverse transcribed into cDNA using, for example, a reverse transcriptase enzyme. Suitable enzymes are commercially available from, for example, Life Technologies/Invitrogen (Carlsbad, Calif.). Optionally, cDNA prepared from mRNA can also be amplified.

### Amplification of Nucleic Acid Molecules

Optionally, the nucleic acid samples obtained from the subject are amplified prior to detection. Target nucleic acids are amplified to obtain amplification products, including a SNP, can be amplified from the sample prior to detection. Typically, DNA sequences are amplified, although in some instances RNA sequences can be amplified or converted into cDNA, such as by using RT PCR.

Any nucleic acid amplification method can be used. An example of in vitro amplification is the polymerase chain reaction (PCR), in which a biological sample obtained from a subject is contacted with a pair of oligonucleotide primers, under conditions that allow for hybridization of the primers to a nucleic acid molecule in the sample. The primers are extended under suitable conditions, dissociated from the template, and then re-annealed, extended, and dissociated to amplify the number of copies of the nucleic acid molecule. Other examples of in vitro amplification techniques include quantitative real-time PCR, strand displacement amplification (see U.S. Pat. No. 5,744,311), transcription-free isothermal amplification (see U.S. Pat. No. 6,033,881), repair chain reaction amplification (see PCT Publication No. WO 90/01069), ligase chain reaction amplification (see EP-A-320 308), gap filling ligase chain reaction amplification (see U.S. Pat. No. 5,427,930), coupled ligase detection and PCR (see U.S. Pat. No. 6,027,889), and NASBA™ RNA transcription-free amplification (see U.S. Pat. No. 6,025,134).

In specific examples, the target sequences to be amplified from the subject include the polymorphic position of at least one SNP selected from the group consisting of rs10733133 (SEQ ID NO: 1), rs1126933 (SEQ ID NO: 2), rs2433031 (SEQ ID NO: 3), rs6571751 (SEQ ID NO: 4), and rs7318267 (SEQ ID NO: 5). In certain embodiments, target sequences containing one or more of SEQ ID NOs: 1-5, or a subset thereof, are amplified. In an embodiment, a single SNP with exceptionally high predictive value is amplified, or a nucleic acid encoding the SNP is amplified.

A pair of primers can be utilized in the amplification reaction. One or both of the primers can be labeled, for example with a detectable radiolabel, fluorophore, or biotin molecule. The pair of primers includes an upstream primer (which binds 5′ to the downstream primer) and a downstream primer (which binds 3′ to the upstream primer). The pair of primers used in the amplification reactions are selective primers which permit amplification of a size related marker locus. Primers can be selected to amplify a nucleic acid comprising the polymorphic position of an SNP. Numerous primers can be designed by those of skill in the art simply by determining the sequence of the desired target region including the polymorphic position of at least one SNP selected from the group consisting of rs10733133 (SEQ ID NO: 1), rs1126933 (SEQ ID NO: 2), rs2433031 (SEQ ID NO: 3), rs6571751 (SEQ ID NO: 4), and rs7318267 (SEQ ID NO: 5), for example, using well known computer assisted algorithms that select primers within desired parameters suitable for annealing and amplification.

If desired, an additional pair of primers can be included in the amplification reaction as an internal control. For example, these primers can be used to amplify a “housekeeping” nucleic acid molecule, and serve to provide confirmation of appropriate amplification. In another example, a target nucleic acid molecule including primer hybridization sites can be constructed and included in the amplification reactor. One of skill in the art will readily be able to identify primer pairs to serve as internal control primers.

### Primer Design Strategy

Increased use of polymerase chain reaction (PCR) methods has stimulated the development of many programs to aid in the design or selection of oligonucleotides used as primers for PCR. Four examples of such programs that are freely available via the Internet are: PRIMER™ by Mark Daly and Steve Lincoln of the Whitehead Institute (UNIX, VMS, DOS, and Macintosh), Oligonucleotide Selection Program by Phil Green and LaDeana Hiller of Washington University in St. Louis (UNIX, VMS, DOS, and Macintosh), PGEN™ by Yoshi (DOS only), and Amplify by Bill Engels of the University of Wisconsin (Macintosh only). Generally these programs help in the design of PCR primers by searching for bits of known repeated-sequence elements and then optimizing the Tₘ by analyzing the length and GC content of a putative primer. Commercial software is also available and primer selection procedures are rapidly being included in most general sequence analysis packages.

Designing oligonucleotides for use as either sequencing or PCR primers requires selection of an appropriate sequence that specifically recognizes the target SNP and its polymorphic position, and then testing the sequence to eliminate the possibility that the oligonucleotide will have a stable secondary structure. Inverted repeats in the sequence can be identified using a repeat-identification or RNA-folding programs. If a possible stem structure is observed, the sequence of the primer can be shifted a few nucleotides in either direction to minimize the predicted secondary structure. When the amplified sequence is intended for subsequence cloning, the sequence of the oligonucleotide can also be compared with the sequences of both strands of the appropriate vector and insert DNA. A sequencing primer only has a single match to the target DNA. It is also advisable to exclude primers that have only a single mismatch with an undesired target DNA sequence. For PCR primers used to amplify genomic DNA, the primer sequence can be compared to the sequences in the GENBANK™ database to determine if any significant matches occur. If the oligonucleotide sequence is present in any known DNA sequence or, more importantly, in any known repetitive elements, the primer sequence should be changed.

### Hybridization

Oligonucleotides and their analogs hybridize by hydrogen bonding, which includes Watson-Crick, Hoogsteen or reversed Hoogsteen hydrogen bonding, between complementary bases. Generally, nucleic acids consist of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence. For example, an oligonucleotide can be complementary to a specific genetic locus, so it specifically hybridizes with a mutant or SNP allele (and not the reference allele) or so that it specifically hybridizes with a reference allele (and not the mutant or SNP allele).

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization. In one example, an oligonucleotide is specifically hybridizable to DNA or RNA nucleic acid sequences including an allele of a gene, wherein it will not hybridize to nucleic acid sequences containing a polymorphism.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, though wash times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11.

The following is an exemplary set of hybridization conditions and is not limiting:

Very High Stringency (Detects Sequences that Share at Least 90% Identity)

Hybridization: 5×SSC at 65° C. for 16 hours; wash twice: 2×SSC at room temperature (RT) for 15 minutes each; and wash twice: 0.5×SSC at 65° C. for 20 minutes each.

High Stringency (Detects Sequences that Share at Least 80% Identity)

Hybridization: 5×-6×SSC at 65° C.-70° C. for 16-20 hours; wash twice: 2×SSC at RT for 5-20 minutes each; and wash twice: 1×SSC at 55° C.-70° C. for 30 minutes each.

Low Stringency (Detects Sequences that Share at Least 50% Identity)

Hybridization: 6×SSC at RT to 55° C. for 16-20 hours and wash at least twice: 2×-3×SSC at RT to 55° C. for 20-30 minutes each.

### Detection of Alleles

The nucleic acids obtained from the sample can be genotyped to identify the particular allele present for a marker locus. A sample of sufficient quantity to permit direct detection of marker alleles from the sample can be obtained from the subject. Alternatively, a smaller sample is obtained from the subject and the nucleic acids are amplified prior to detection. Any nucleic acid that is informative for an SNP of the present invention can be detected. Generally, the target nucleic acid comprises the polymorphic position of an SNP selected from the group consisting of rs10733133 (SEQ ID NO: 1), rs1126933 (SEQ ID NO: 2), rs2433031 (SEQ ID NO: 3), rs6571751 (SEQ ID NO: 4), and rs7318267 (SEQ ID NO: 5). Any method of detecting a nucleic acid molecule can be used, such as hybridization and/or sequencing assays.

Hybridization is the binding of complementary strands of DNA, DNA/RNA, or RNA. Hybridization can occur when primers or probes bind to target sequences such as target sequences within genomic DNA. Probes and primers that are useful generally include nucleic acid sequences that hybridize (for example under high stringency conditions) with a nucleic acid sequence including a SNP of interest, but do not hybridize to a reference allele, or that hybridize to the reference allele, but do not hybridize to the SNP. Physical methods of detecting hybridization or binding of complementary strands of nucleic acid molecules, include but are not limited to, such methods as DNase I or chemical footprinting, gel shift and affinity cleavage assays, Southern and Northern blotting, dot blotting, and light absorption detection procedures. The binding between a nucleic acid primer or probe and its target nucleic acid is frequently characterized by the temperature (Tₘ) at which 50% of the nucleic acid probe is melted from its target. A higher Tₘ means a stronger or more stable complex relative to a complex with a lower Tₘ.

Generally, complementary nucleic acids form a stable duplex or triplex when the strands bind, (hybridize), to each other by forming Watson-Crick, Hoogsteen or reverse Hoogsteen base pairs. Stable binding occurs when an oligonucleotide molecule remains detectably bound to a target nucleic acid sequence under the required conditions.

Complementarity is the degree to which bases in one nucleic acid strand base pair with the bases in a second nucleic acid strand. Complementarity is conveniently described by percentage, that is, the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between an oligonucleotide molecule and a target nucleic acid sequence (such as an SNP) to achieve detectable and specific binding. When expressed or measured by percentage of base pairs formed, the percentage complementarity that fulfills this goal can range from as little as about 50% complementarity to full (100%) complementary. In general, sufficient complementarity is at least about 50%, for example at least about 75% complementarity, at least about 90% complementarity, at least about 95% complementarity, at least about 98% complementarity, or even at least about 100% complementarity. The qualitative and quantitative considerations involved in establishing binding conditions that allow one skilled in the art to design appropriate oligonucleotides for use under the desired conditions is provided by Beltz et al. Methods Enzymol 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning: A Laboratory Manual, second edition, Cold Spring Harbor Laboratory, Plainview, N.Y. (chapters 9 and 11). Exemplary hybridization conditions are provided above.

Methods for labeling nucleic acid molecules so they can be detected are well known. Examples of such labels include non-radiolabels and radiolabels. Non-radiolabels include, but are not limited to an enzyme, chemiluminescent compound, fluorescent compound (such as FITC, Cy3, and Cy5), metal complex, hapten, enzyme, colorimetric agent, a dye, or combinations thereof. Radiolabels include, but are not limited to, ¹²⁵I, ³²P and ³⁵S. For example, radioactive and fluorescent labeling methods, as well as other methods known in the art, are suitable for use with the present disclosure. In one example, primers used to amplify the subject's nucleic acids are labeled (such as with biotin, a radiolabel, or a fluorophore). In another example, amplified target nucleic acid samples are end-labeled to form labeled amplified material. For example, amplified nucleic acid molecules can be labeled by including labeled nucleotides in the amplification reactions.

Nucleic acid molecules comprising one or more polymorphic positions of an SNP selected from the group consisting of rs10733133 (SEQ ID NO: 1), rs1126933 (SEQ ID NO: 2), rs2433031 (SEQ ID NO: 3), rs6571751 (SEQ ID NO: 4), and rs7318267 (SEQ ID NO: 5) can also be detected by hybridization procedures using a labeled nucleic acid probe, such as a probe that detects only one alternative allele at a marker locus. Most commonly, the target nucleic acid (or amplified target nucleic acid) is separated based on size or charge and transferred to a solid support. The solid support (such as membrane made of nylon or nitrocellulose) is contacted with a labeled nucleic acid probe, which hybridizes to it complementary target under suitable hybridization conditions to form a hybridization complex.

Hybridization conditions for a given combination of array and target material can be optimized routinely in an empirical manner close to the Tₘ of the expected duplexes, thereby maximizing the discriminating power of the method. For example, the hybridization conditions can be selected to permit discrimination between matched and mismatched oligonucleotides. Hybridization conditions can be chosen to correspond to those known to be suitable in standard procedures for hybridization to filters (and optionally for hybridization to arrays). In particular, temperature is controlled to substantially eliminate formation of duplexes between sequences other than an exactly complementary allele of the selected marker. A variety of known hybridization solvents can be employed, the choice being dependent on considerations known to one of skill in the art (see U.S. Pat. No. 5,981,185).

Once the target nucleic acid molecules have been hybridized with the labeled probes, the presence of the hybridization complex can be analyzed, for example by detecting the complexes.

Methods for detecting hybridized nucleic acid complexes are well known in the art. In one example, detection includes detecting one or more labels present on the oligonucleotides, the target (e.g., amplified) sequences, or both. Detection can include treating the hybridized complex with a buffer and/or a conjugating solution to effect conjugation or coupling of the hybridized complex with the detection label, and treating the conjugated, hybridized complex with a detection reagent. In one example, the conjugating solution includes streptavidin alkaline phosphatase, avidin alkaline phosphatase, or horseradish peroxidase. Specific, non-limiting examples of conjugating solutions include streptavidin alkaline phosphatase, avidin alkaline phosphatase, or horseradish peroxidase. The conjugated, hybridized complex can be treated with a detection reagent. In one example, the detection reagent includes enzyme-labeled fluorescence reagents or calorimetric reagents. In one specific non-limiting example, the detection reagent is enzyme-labeled fluorescence reagent (ELF) from Molecular Probes, Inc. (Eugene, Oreg.). The hybridized complex can then be placed on a detection device, such as an ultraviolet (UV) transilluminator (manufactured by UVP, Inc. of Upland, Calif.). The signal is developed and the increased signal intensity can be recorded with a recording device, such as a charge coupled device (CCD) camera. In particular examples, these steps are not performed when radiolabels are used. In particular examples, the method further includes quantification, for instance by determining the amount of hybridization.

### Allele Specific PCR

Allele-specific PCR differentiates between target regions differing in the presence of absence of a variation or polymorphism. PCR amplification primers are chosen based upon their complementarity to a nucleic acid sequence, such as nucleic acid sequence in a SNP or a specified region of an allele including an SNP. The primers bind only to certain alleles of the target sequence. This method is described by Gibbs, Nucleic Acid Res. 17:12427 2448, 1989, herein incorporated by reference.

### Allele Specific Oligonucleotide Screening Methods

Further screening methods employ the allele-specific oligonucleotide (ASO) screening methods (e.g. see Saiki et al., Nature 324:163-166, 1986). Oligonucleotides with one or more base pair mismatches are generated for any particular allele. ASO screening methods detect mismatches between one allele in the target genomic or PCR amplified DNA and the other allele showing decreased binding of the oligonucleotide relative to the second allele (e.g., the other allele) oligonucleotide. Oligonucleotide probes can be designed that under low stringency will bind to both polymorphic forms of the allele, but which at high stringency, only bind to the allele to which they correspond. Alternatively, stringency conditions can be devised in which an essentially binary response is obtained. For example, an ASO corresponding to a variant form of the target gene will hybridize to that allele (haplotype block), and not to the reference allele (haplotype block).

### Ligase Mediated Allele Detection Method

Ligase can also be used to detect point mutations, such as the SNPs disclosed herein, in a ligation amplification reaction (e.g. as described in Wu et al., Genomics 4:560-569, 1989). The ligation amplification reaction (LAR) utilizes amplification of specific DNA sequence using sequential rounds of template dependent ligation (e.g., as described in Barany, Proc. Nat. Acad. Sci. 88:189-193, 1990).

### Denaturing Gradient Gel Electrophoresis

Amplification products generated using the polymerase chain reaction can be analyzed by the use of denaturing gradient gel electrophoresis. Different SNPs or alleles can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (Tₘ). Melting domains are at least 20 base pairs in length, and can be up to several hundred base pairs in length.

Differentiation between SNPs or alleles based on sequence specific melting domain differences can be assessed using polyacrylamide gel electrophoresis, as described in Chapter 7 of Erlich, ed., PCR Technology, Principles and Applications for DNA Amplification, W. H. Freeman and Co., New York (1992).

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527, 1986, and Myers et al., in Genomic Analysis, A Practical Approach, K. Davies Ed. IRL Press Limited, Oxford, pp. 95 139, 1988. The electrophoresis system is maintained at a temperature slightly below the Tₘ of the melting domains of the target sequences.

In an alternative method of denaturing gradient gel electrophoresis, the target sequences can be initially attached to a stretch of GC nucleotides, termed a GC clamp, as described in Chapter 7 of Erlich, supra. In one example, at least 80% of the nucleotides in the GC clamp are either guanine or cytosine. In another example, the GC clamp is at least 30 bases long. This method is particularly suited to target sequences with a high Tₘ.

Generally, the target region is amplified by polymerase chain reaction. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions. DNA fragments differing by a single base change will migrate through the gel to different positions, which can be visualized by ethidium bromide staining.

### Temperature Gradient Gel Electrophoresis

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

### Single-Strand Conformation Polymorphism Analysis

Target sequences, such as alleles, can be differentiated using single-strand conformation polymorphism analysis, which identifies base differences by alteration in electrophoretic migration of single stranded PCR products, for example as described in Orita et al., Proc. Nat. Acad. Sci. 85:2766-2770, 1989. Amplified PCR products can be generated as described above, and heated or otherwise denatured, to form single stranded amplification products. Single-stranded nucleic acids can refold or form secondary structures which are partially dependent on the base sequence. Thus, electrophoretic mobility of single-stranded amplification products can detect base-sequence difference between alleles or haplotype blocks.

### Chemical or Enzymatic Cleavage of Mismatches

Differences between target sequences, such as alleles, can also be detected by differential chemical cleavage of mismatched base pairs, for example as described in Grompe et al., Am. J. Hum. Genet. 48:212-222, 1991. In another method, differences between target sequences, such as alleles, can be detected by enzymatic cleavage of mismatched base pairs, as described in Nelson et al., Nature Genetics 4:11-18, 1993.

### Non-Gel Systems

Other possible techniques include non-gel systems such as TaqMan™ (Perkin Elmer). In this system oligonucleotide PCR primers are designed that flank the allele in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete (there is a mismatch of some form) the cleavage of the dye does not take place. Thus only if the nucleotide sequence of the oligonucleotide probe is completely complimentary to the template molecule to which it is bound will quenching be removed. A reaction mix can contain two different probe sequences each designed against different alleles that might be present thus allowing the detection of both alleles in one reaction.

### Non-PCR Based Allele Detection

The identification of a DNA sequence can be made without an amplification step, based on polymorphisms including restriction fragment length polymorphisms in a subject and a control, such as a family member or unaffected individual. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes can be labeled directly or indirectly, such that by assaying for the presence or absence of the probe, one can detect the presence or absence of the target sequence. Direct labeling methods include radioisotope labeling, such as with ³²P or ³⁵S. Indirect labeling methods include fluorescent tags, biotin complexes which can be bound to avidin or streptavidin, or peptide or protein tags. Visual detection methods include photoluminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to a nucleic acid sequence wherein a polymorphism is present, thus defining a genetic marker, including a restriction fragment length polymorphism, a hypervariable region, repetitive element, or a variable number tandem repeat. Hybridization probes can be any gene or a suitable analog. Further suitable hybridization probes may include exon fragments or portions of cDNAs or genes known to map to the relevant region of the chromosome.

The presence or absence of a polymorphism can also be determined using one or both chromosomal complements represented in the nucleic acid sample. Determining the presence or absence of a polymorphic variant in both chromosomal complements represented in a nucleic acid sample is useful for determining the zygosity of an individual for the polymorphic variant (i.e., whether the individual is homozygous or heterozygous for the polymorphic variant). Any oligonucleotide-based diagnostic may be utilized to determine whether a sample includes the presence or absence of a polymorphic variant in a sample. For example, primer extension methods, ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), microarray sequence determination methods, restriction fragment length polymorphism (RFLP), single strand conformation polymorphism detection (SSCP) (e.g., U.S. Pat. Nos. 5,891,625 and 6,013,499), PCR-based assays (e.g., TAQMAN™ PCR System (Applied Biosystems)), and nucleotide sequencing methods may be used.

### Kits and Apparatuses

An apparatus for detecting a nucleotide in a nucleic acid sequence is provided. Typically, the apparatus comprises a substrate, such as a glass slide, and at least two oligonucleotides bound to the substrate. In certain embodiments, the apparatus comprises at least one oligonucleotide, at least two oligonucleotides, at least three oligonucleotides, at least four oligonucleotides, at least five oligonucleotides, at least six oligonucleotides, at least seven oligonucleotides, at least eight oligonucleotides, at least nine oligonucleotides, or at least ten oligonucleotides, wherein each of the oligonucleotides comprises a contiguous nucleic acid sequence complementary to a different sequence selected from group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15 shown in Table 3.

In some embodiments, the apparatus comprises one or more oligonucleotides wherein each of the oligonucleotides comprises a contiguous nucleic acid sequence selected from SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

TABLE 3
Oligonucleotides comprising a contiguous nucleic
acid sequence complementary to any one of SEQ ID
NOs: 6-15 and/or oligonucleotides comprising a
contiguous nucleic acid sequence corresponding
to any one of SEQ ID NOs: 6-15 may be coupled to
a substrate in an apparatus such as a microarray.

| SEQ | |
|---|---|
| ID NO: | Nucleotide Sequence |
| 6 | GAAAGCATACTTGGCCAAATTTTCAACTGATTCCCTGTCATT |
| | AGCAAATATG |
| 7 | ATCTGCAACCAAAGCCCTGTGCGCCACGTGCTGAACATCAC |
| | GGACAAGGACC |
| 8 | TTAGAACACTCTGTATTAAGCCAGCAATGTGAGTATAAAATG |
| | ATTTCTAAAT |
| 9 | ATGAAAACAGAGGTGAAGTAGCCAGTAAACCCAGTGAACTG |
| | TGAGTTCTTCT |
| 10 | GTCCTCATCCTCCTTCCTCAAAGCCACCGGCTCCTCCTGGA |
| | CGGGTTGGGTA |
| 11 | GAAAGCATACTTGGCCAAATTTTCAATTGATTCCCTGTCATT |
| | AGCAAATATG |
| 12 | ATCTGCAACCAAAGCCCTGTGCGCCAGGTGCTGAACATCAC |
| | GGACAAGGACC |
| 13 | TTAGAACACTCTGTATTAAGCCAGCATTGTGAGTATAAAATG |
| | ATTTCTAAAT |
| 14 | ATGAAAACAGAGGTGAAGTAGCCAGTGAACCCAGTGAACT |
| | GTGAGTTCTTCT |
| 15 | GTCCTCATCCTCCTTCCTCAAAGCCATCGGCTCCTCCTGGA |
| | CGGGTTGGGTA |

Although the length of the oligonucleotides for use with the apparatus can be chosen in part based on the overall characteristics of the oligonucleotides on the substrate, a preferred range of lengths are between 25-mer and 60-mer.

A microarray can be utilized for determining whether the polymorphism is present or absent in a nucleic acid sample. A microarray may include any oligonucleotides described hereinabove. Methods for making and using oligonucleotide microarrays suitable for diagnostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; and WO 01/29259. The microarray typically comprises a solid support and the oligonucleotides may be linked to this solid support by covalent bonds or by non-covalent interactions. The oligonucleotides may also be linked to the solid support directly or by a spacer molecule. A microarray may comprise one or more oligonucleotides complementary to a polymorphism.

A kit also may be utilized for determining whether a polymorphism is present or absent in a nucleic acid sample. A kit can include one or more pairs of oligonucleotide primers useful for amplifying a fragment of a nucleotide sequence of interest, where the fragment includes a polymorphic site. The kit may comprise a polymerizing agent, for example, a thermostable nucleic acid polymerase such as one disclosed in U.S. Pat. No. 4,889,818 or 6,077,664. The polymerizing agent may be a DNA polymerase, RNA polymerase or reverse transcriptase. The kit may also comprise an elongation oligonucleotide that hybridizes to the nucleotide sequence in a nucleic acid sample adjacent to the polymorphic site. The kit can also include chain elongating nucleotides, such as dATP, dTTP, dGTP, dCTP, and dITP, including analogs of dATP, dTTP, dGTP, dCTP and dITP, provided that such analogs are substrates for a thermostable nucleic acid polymerase and can be incorporated into a nucleic acid chain elongated from the extension oligonucleotide. Along with chain elongating nucleotides would be one or more chain terminating nucleotides such as ddATP, ddTTP, ddGTP, ddCTP. The kit can include one or more oligonucleotide primer pairs, a polymerizing agent, chain elongating nucleotides, at least one elongation oligonucleotide, and one or more chain terminating nucleotides. Kits optionally include buffers, vials, microtiter plates, and instructions for use.

The present invention is further illustrated by the following examples that should not be construed as limiting. The contents of all references, patents, and published patent applications cited throughout this application, as well as the FIGURE, are incorporated herein by reference in their entirety for all purposes.

### EXAMPLES

The use of rhBMP-2 (Medtronic, Memphis, Tenn.) in spinal fusion surgeries remains controversial due to off-label use, cost, and reported complications despite high fusion rates. Up to 4.6% of rhBMP-2 posterolateral lumbar arthrodeses have a complication of postoperative painful seroma formation requiring secondary surgery. The biological attributes underlying these responses to rhBMP-2 have not been fully elucidated. This study aims to identify genetic variants that will predict a patient's risk of complications associated with rhBMP-2.

All of the analyzed groups of patients underwent spinal fusion surgery and received rhBMP-2. The group of “hyporesponders” either had limited bone growth or delayed healing or required secondary surgery due to failed fusion. The group of “normal responders” had the expected, successful result of the surgery, and the group of “non-hyperresponders” was made up of both the “hyporesponders” and the “normal responders”. The group of “hyperresponders” experienced a hyper reaction resulting from the rhBMP-2 administration and suffered from such complications as seroma formation, hyperemia induced swelling, and bone overgrowth.

The exomes of six hyper-responders and two hypo-responders were used to identify potential single nucleotide polymorphisms (SNPs) which differentiated the cohorts. The exomes were indexed and hybridized (Agilent's SureSelect Illumina multiplex paired end protocol) and then sequenced (Illumina HiSeq 2000). A total of 18.8 billion high quality bases were called, 98.4% of which aligned to the reference genome with an average error rate of 0.3% per sample. Raw reads from HiSeq were converted to standard format using Illumina provided software and custom scripts. Bar coded reads were split and all reads for each sample were collected together. Reads were aligned to reference human genome (build 36) using BWA aligner. Alignments were processed to remove duplicates, recalibrate the quality scores, and sorted using custom scripts and Picard and GATK software tools. Processed alignments were used for variant calling involving two calling algorithms—samtools and varscan. Only those variants in coding regions that were picked up by both callers were retained and annotated with gene names and other attributes. Of the SNPs identified, 205 were found in at least four of the hyper-responders but zero of the hypo-responders. These top hits were then prioritized according to specific molecular pathways and known genes related to bone growth and development and genotyped using the Sequenom MassArray platform on an additional validation set consisting of 46 hypo-, 24 hyper-, and 52 normal-responders. Standard statistical analyses of genetic association were performed to identify SNPs that correlate with hyperresponse to rhBMP-2 using statistical modeling techniques similar to those described in Zheng et al., N Engl J Med (2008) 358:910-919. The results are presented in Table 4 and Table 5.

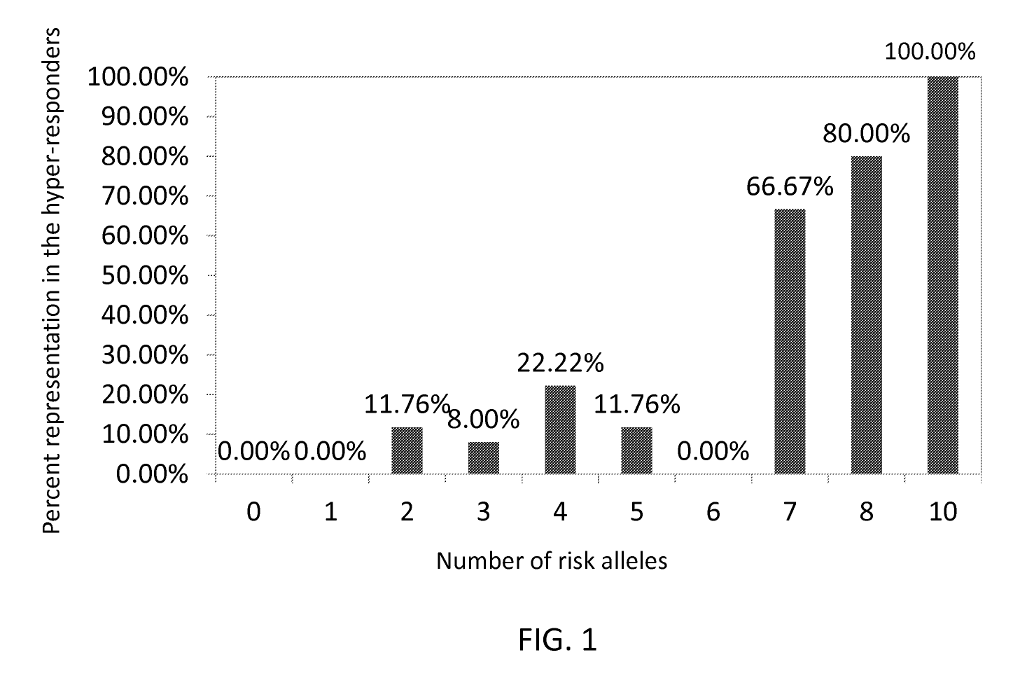

An allelic test comparing frequency of each SNP between hyperresponders and non-hyperresponders confirmed an association for 5 SNPs (p<0.05); these consisted of nonsynonymous changes or splice variants in NID1, SENP7, FARP1, RPGRIP1, and CDH3 (see Table 2). The performance of each mutation for discriminating hyperresponders from non-hyperresponders, measured by area under the receiver operating characteristic curves (AUC), ranged from 0.65 to 0.68. The combined AUC of these 5 variants was 0.75 (p=0.0003). These results are presented in Table 6. Among the 108 Caucasian patients treated with rhBMP-2, 78% of those with 7 or more hyperresponsive alleles were hyperresponders; in comparison, only 10% were hyperresponders among those patients with 6 or fewer hyperresponsive alleles, as illustrated in FIG. 1. Importantly, differentiation between the two groups was highly significant (p=0.00002).

These results are the first to suggest that a combination of multiple genetic variants associated with pathological response to rhBMP-2 can be used to identify those at greatest risk to be hyperresponders. Studies to validate the predictive power of the current panel of SNPs in independent cohorts of rhBMP-2 treated patients will be performed. This study provides the foundation to support pre-operative genetic screening to guide rhBMP-2 use in spinal arthrodesis patients.

TABLE 4
Statistical significance of the five SNPs in distinguishing hyperresponders
to rhBMP-2 treatment from non-hyperresponders.

| | | | | Non- | | |
|---|---|---|---|---|---|---|
| | | | Hyper_A1 | Hyper_A1 | | Odds |
| SNP | A1 | A2 | Frequency | Frequency | p-value | Ratio |
| rs10733133 | T | C | 0.6562 | 0.3929 | 0.005877 | 2.95 |
| rs1126933 | C | G | 0.5667 | 0.3476 | 0.02311 | 2.455 |
| rs2433031 | T | A | 0.5312 | 0.321 | 0.02318 | 2.397 |
| rs6571751 | A | G | 0.2857 | 0.529 | 0.01887 | 0.3562 |
| rs7318267 | T | C | 0.5312 | 0.3171 | 0.02032 | 2.441 |

TABLE 5
Statistical significance of the five SNPs in distinguishing hyperresponders
to rhBMP-2 treatment from hyporesponders.

| | | | Hyper_A1 | Hypo_A1 | | Odds |
|---|---|---|---|---|---|---|
| SNP | A1 | A2 | Frequency | Frequency | p-value | Ratio |
| rs10733133 | T | C | 0.6562 | 0.3472 | 0.003405 | 3.589 |
| rs1126933 | C | G | 0.5667 | 0.3108 | 0.01511 | 2.9 |
| rs2433031 | T | A | 0.5312 | 0.2794 | 0.01439 | 2.923 |
| rs6571751 | A | G | 0.2857 | 0.5667 | 0.01398 | 0.3059 |
| rs7318267 | T | C | 0.5312 | 0.3143 | 0.03642 | 2.473 |

TABLE 6
SNPs that confer risk of hyper-response to rhBMP-2 as measure
by area under the operating characteristic curves (AUC) analysis

| SNP | Gene Name | Chi-Square | p value | AUC |
|---|---|---|---|---|
| rs10733133 | NID1 | 2.0042 | 1.57E−01 | 0.68 |
| rs1126933 | CDH3 | 1.2857 | 2.57E−01 | 0.663 |
| rs2433031 | SENP7 | 1.4023 | 2.36E−01 | 0.665 |
| rs6571751 | RPGRIP1 | 2.0647 | 1.151E−01 | 0.663 |
| rs7318267 | FARP1 | 3.1426 | 7.63E−02 | 0.652 |
| All | | | 0.0003 | 0.749 |

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A method of predicting an increased likelihood of a complication associated with administration of a bone morphogenetic protein (BMP) in a human subject in need of a spinal fusion surgery comprising the steps of:
obtaining a sample from the human subject in need of the spinal fusion surgery;
detecting in the sample the presence of one or a combination of two or more single nucleotide polymorphisms (SNPs) selected from the group consisting of a thymine at the polymorphic position of rs10733133 (SEQ ID NO: 1), a cytosine at the polymorphic position of rs1126933 (SEQ ID NO: 2), a thymine at the polymorphic position of rs2433031 (SEQ ID NO: 3), a guanine at the polymorphic position of rs6571751 (SEQ ID NO: 4), and a thymine at the polymorphic position of rs7318267 (SEQ ID NO: 5); and
predicting the human subject with the one or combination of SNPs has an increased likelihood of experiencing a complication resulting from administration of a BMP compared to a human subject without the one or combination of SNPs.

2. The method of claim 1 further comprising the steps of:
amplifying a nucleic acid sequence comprising the polymorphic position of an SNP selected from the group consisting of rs10733133 (SEQ ID NO: 1), rs1126933 (SEQ ID NO: 2), rs2433031 (SEQ ID NO: 3), rs6571751 (SEQ ID NO: 4), and rs7318267 (SEQ ID NO: 5) using a first primer that binds upstream of said position and a second primer that binds downstream of said position; and
determining the genotype of the human subject at said position.

3. The method according to claim 2, wherein the complication results from a hyperresponse to the BMP.

4. The method of claim 3, wherein the complication is selected from the group consisting of seroma formation, painful edema, compressive fluid collection, bone overgrowth, and hyperemia induced swelling.

5. The method of claim 1, wherein the spinal fusion surgery is a posterolateral fusion or an interbody fusion.

6. The method of claim 5, wherein the interbody fusion is selected from the group consisting of anterior lumbar interbody fusion (ALIF), posterior lumbar interbody fusion (PLIF), transforaminal lumbar interbody fusion (TLIF), transpsoas interbody fusion (DLIF or XLIF), and cervical interbody fusion.

7. The method of claim 2, wherein the human subject is homozygous for at least one of the SNPs.

8. The method of claim 7, wherein detecting in the sample six or more alleles with the SNPs indicates an increased likelihood of the human subject experiencing a complication associated with administration of a BMP compared to a human subject without the SNPs.

9. The method of claim 8, wherein detecting in the sample seven or more alleles with the SNPs indicates an increased likelihood of the human subject experiencing a complication associated with administration of a BMP compared to a human subject without the SNPs.

10. The method of claim 1, wherein the BMP is recombinant human bone morphogenetic protein-2 (rhBMP-2) or recombinant human bone morphogenetic protein-7 (rhBMP-7).

11. A method of treating a human subject in need of a spinal fusion surgery comprising:
obtaining a sample from the human subject in need of the spinal fusion surgery;
detecting in the sample the absence of one or a combination of two or more SNPs selected from the group consisting of a thymine at the polymorphic position of rs10733133, a cytosine at the polymorphic position of rs1126933, a thymine at the polymorphic position of rs2433031, a guanine at the polymorphic position of rs6571751, and a thymine at the polymorphic position of rs7318267, wherein the absence of the one or combination of SNPs indicates a decreased likelihood of experiencing a complication associated with administration of a BMP compared to a human subject with the one or combination of SNPs; and
administering a BMP to the human subject.

12. The method of claim 11 further comprising the steps of:
amplifying a nucleic acid sequence comprising the polymorphic position of an SNP selected from the group consisting of rs10733133 (SEQ ID NO: 1), rs1126933 (SEQ ID NO: 2), rs2433031 (SEQ ID NO: 3), rs6571751 (SEQ ID NO: 4), and rs7318267 (SEQ ID NO: 5) using a first primer that binds upstream of said position and a second primer that binds downstream of said position; and
determining the genotype of the human subject at said position.

13. The method of claim 12, wherein the complication results from a hyperresponse to the BMP.

14. The method of claim 13, wherein the complication is selected from the group consisting of seroma formation, painful edema, compressive fluid collection, bone overgrowth, and hyperemia induced swelling.

15. The method of claim 11, wherein the spinal fusion surgery is a posterolateral fusion or an interbody fusion.

16. The method of claim 15, wherein the interbody fusion is selected from the group consisting of anterior lumbar interbody fusion (ALIF), posterior lumbar interbody fusion (PLIF), transforaminal lumbar interbody fusion (TLIF), transpsoas interbody fusion (DLIF or XLIF), and cervical interbody fusion.

17. The method of claim 16, wherein the BMP is rhBMP-2 or rhBMP-7.

18. A method of treating a human subject in need of a spinal fusion surgery comprising:
obtaining a sample from the human subject in need of a spinal fusion surgery;
detecting in the sample the presence of one or a combination of two or more SNPs selected from the group consisting of a cytosine at the polymorphic position of rs10733133, a guanine at the polymorphic position of rs1126933, an adenine at the polymorphic position of rs2433031, an adenine at the polymorphic position of rs6571751, and a cytosine at the polymorphic position of rs7318267, wherein the presence of the one or combination of SNPs indicates a decreased likelihood of experiencing a complication associated with administration of a BMP compared to a human subject with the one or combination of SNPs; and
administering a BMP to the human subject.

19. The method of claim 18 further comprising the steps of:
amplifying a nucleic acid sequence containing position 27 of an SNP selected from the group consisting of rs10733133 (SEQ ID NO: 1), rs1126933 (SEQ ID NO: 2), rs2433031 (SEQ ID NO: 3), rs6571751 (SEQ ID NO: 4), and rs7318267 (SEQ ID NO: 5) using a first primer that binds upstream of said position and a second primer that binds downstream of said position; and
determining the genotype of the human subject at said position.

20. The method of claim 19, wherein the BMP is rhBMP-2 or rhBMP-7.

21.-25. (canceled)


SEQUENCE LISTING (15 sequences)

SEQ ID NO: 1 - DNA, 52 bp, Homo sapiens
  misc_feature (27)..(27): n is c or t
gaaagcatacttggccaaattttcaantgattccctgtcattagcaaatatg

SEQ ID NO: 2 - DNA, 52 bp, Homo sapiens
  misc_feature (27)..(27): n is c or g
atctgcaaccaaagccctgtgcgccangtgctgaacatcacggacaaggacc

SEQ ID NO: 3 - DNA, 52 bp, Homo sapiens
  misc_feature (27)..(27): n is a or t
ttagaacactctgtattaagccagcantgtgagtataaaatgatttctaaat

SEQ ID NO: 4 - DNA, 52 bp, Homo sapiens
  misc_feature (27)..(27): n is a or g
atgaaaacagaggtgaagtagccagtnaacccagtgaactgtgagttcttct

SEQ ID NO: 5 - DNA, 52 bp, Homo sapiens
  misc_feature (27)..(27): n is c or t
gtcctcatcctccttcctcaaagccancggctcctcctggacgggttgggta

SEQ ID NO: 6 - DNA, 52 bp, Artificial Sequence
  Oligonucleotide
gaaagcatacttggccaaattttcaactgattccctgtcattagcaaatatg

SEQ ID NO: 7 - DNA, 52 bp, Artificial Sequence
  Oligonucleotide
atctgcaaccaaagccctgtgcgccacgtgctgaacatcacggacaaggacc

SEQ ID NO: 8 - DNA, 52 bp, Artificial Sequence
  Oligonucleotide
ttagaacactctgtattaagccagcaatgtgagtataaaatgatttctaaat

SEQ ID NO: 9 - DNA, 52 bp, Artificial Sequence
  Oligonucleotide
atgaaaacagaggtgaagtagccagtaaacccagtgaactgtgagttcttct

SEQ ID NO: 10 - DNA, 52 bp, Artificial Sequence
  Oligonucleotide
gtcctcatcctccttcctcaaagccaccggctcctcctggacgggttgggta

SEQ ID NO: 11 - DNA, 52 bp, Artificial Sequence
  Oligonucleotide
gaaagcatacttggccaaattttcaattgattccctgtcattagcaaatatg

SEQ ID NO: 12 - DNA, 52 bp, Artificial Sequence
  Oligonucleotide
atctgcaaccaaagccctgtgcgccaggtgctgaacatcacggacaaggacc

SEQ ID NO: 13 - DNA, 52 bp, Artificial Sequence
  Oligonucleotide
ttagaacactctgtattaagccagcattgtgagtataaaatgatttctaaat

SEQ ID NO: 14 - DNA, 52 bp, Artificial Sequence
  Oligonucleotide
atgaaaacagaggtgaagtagccagtgaacccagtgaactgtgagttcttct

SEQ ID NO: 15 - DNA, 52 bp, Artificial Sequence
  Oligonucleotide
gtcctcatcctccttcctcaaagccatcggctcctcctggacgggttgggta